(19) United States
(12) Patent Application Publication
(10) Pub. No.: US 2019/0006040 A1
(43) Pub. Date: Jan. 3, 2019

(54) COGNITIVE DIABETIC REGULATOR

(71) Applicant: International Business Machines Corporation, Armonk, NY (US)

(72) Inventors: KALA FLEMING, NAIROBI (KE); SALLY SIMONE FLORE ROSE LYLIE FOBI NSUTEZO, NAIROBI (KE); MICHAEL GORDON, YORKTOWN HEIGHTS, NY (US); KOMMINIST WELDEMARIAM, NAIROBI (KE)

(21) Appl. No.: 15/859,452

(22) Filed: Dec. 30, 2017

Related U.S. Application Data
(63) Continuation of application No. 15/640,509, filed on Jul. 1, 2017.

(51) Int. Cl. G09B 19/00; G06Q 10/10

(52) U.S. Cl. CPC G16H 20/60; G06Q 10/10; G16H 10/60; G09B 19/0092

(57) ABSTRACT

The disclosure provides systems and methods for determining, customizing, and communicating dietary recommendations, particularly for patients suffering from Type 2 diabetes. The system includes data gathered via sensors configured to sense activities and contextual information pertaining to a user, modules for determining the diabetic state and risk pertaining to the user, and meal planning and context analysing modules for determining appropriate recommendations for the user. Determined meals, recommended activities, and other outputs are communicated to the user.

### CROSS-REFERENCE TO RELATED APPLICATIONS

This application is a continuation of U.S. patent application Ser. No. 15/640,509 filed Jul. 1, 2017, the complete disclosure of which is expressly incorporated herein by reference in its entirety for all purposes.

### BACKGROUND

In embodiments, the technical field of the invention is methods and systems for determining, customizing, and communicating dietary recommendations.

Diabetes mellitus (Type 2) is a blood glucose regulatory condition that occurs due to the absence or improper use of insulin in the body. It affects approximately 6% of the world's adult population, of which 80% of diabetics are in developing regions. In Africa alone, there were 7 million diabetics in 2000 and the number is expected to reach 18 millions by 2030. In fact, some developing populations show higher incidences of diabetes (roughly double the global incidence rate). Unmanaged Type 2 diabetes can lead to fatigue, weight change, nerve damage, decreased vision, kidney problems, strokes, amputations and even death.

Blood Glucose has traditionally been the best indicator for diabetes diagnoses. Two glucoses tests can be used to estimate the instantaneous blood-sugar level: Fasting Plasma Glucose (FPG) and Oral Glucose Tolerance Test (OGTT). A more robust indicator is the A1C or glycohemoglobin test, which describes glucose over a long period time. These tests are inconvenient and, in many locations, unavailable or unreliably available.

Medication is used to manage massive fluctuations in blood glucose level. Low glucose diets are used to reduce blood sugar levels. Exercise and weight loss is also used to decrease blood sugar levels. Although the link between diabetes and food is not obvious nutritional choices affect the blood sugar level. Glycemic Index (GI) reflects the effect of a particular food on a person's blood sugar level. At a value of 100, the food is equivalent to pure glucose. GI shows total increase in glucose in the blood but not the rate of increase. Foods with high GI raise blood sugar level more than food with low GI. It is known that appropriate nutritional choices can minimize substantial spikes in blood sugar levels, reducing the negative effects of diabetes.

Many problems are associated with current methods of diagnosing, treating, and managing diabetes. Invasive blood glucose monitors are painful and non-invasive monitors are unreliable as well as context dependant. Blood glucose monitoring is primarily done through invasive methods including, finger pricks and sub-cutaneous devices or sensors. These can be painful and uncomfortable. Non-invasive monitors include determining blood glucose from retina, saliva, breath and skin have mostly proven elusive and unreliable. Currently, Meal Plan Recommenders target weight loss over comprehensive diabetes management. Meal planners are not tailored to diabetes management. They do not use data from sensory devices to provide nutrition advice for diabetics. They focus mostly on weight loss through calorie management rather than sugar management. Meal Plan Recommenders do not consider the user's budget, seasonality of foods, allergies, or proximity to market. Furthermore, they often do not account for dietary restrictions due to allergies, meal preferences, religious beliefs, etc.

There is currently no comprehensive system that monitors risk level of a patient, recommends appropriate meals and gauges sentiment to provide appropriate support/encouragement. There is also no comprehensive system that monitors/predicts risk level for a diabetic, and recommends meals where the ingredients are within proximity, affordable and to their preference, offering holistic diabetes management.

### SUMMARY

In an aspect, this invention provides an intelligent meal planner (MP) that generates optimal meal plans and other recommendations to regulate the diabetic condition of a patient. As described herein in detail, the MP uses speech, gait, expression, blood glucose, facial expression, and possibly other factors (individually or in combination) to predict the risk level of a diabetic and, based on the risk level, recommends optimal meals, recipes, activities, and the like. Detection and/or prediction of a patient's diabetic risk level is based on patient history and analysis (e.g., using deep learning and visual analysis) of the patient's speech, facial expression, heart rate, etc. captured via one or more sensors on a user device. The device may be a stand-alone sensor, or may be a device comprising a sensor, such as a wearable device, mobile, tablet, camera, etc. The sensor is instrumented to collect data in a non-invasive or minimally invasive manner.

In addition to diabetic risk, the optimal time needed to adjust the patient's current diabetic state can be determined. The current state of the patient is influenced by the risk level, patient nutrition, patient medication list, and time required to stabilize the patient, among other potential factors. Given the determined current state, an amelioration action (or more than one such actions) will be triggered and shown through visual or other indicators—e.g., a user interface exhibiting changing display color and intensity. The one or more amelioration actions may further include, for example, automatically calling family or close friends, which may be identified from the person's call logs or social media to identify suitable person(s) to call. The identification a family member or close friend from the person's call logs is based on social network analysis which may use machine learning or graph analysis algorithms, among other possible methods. Other examples of amelioration actions may include a recommendation to drink a cup of orange juice or other available energy source at an identified retail store closest to the patient based on proximity analysis.

Given the patient's determined current state, the MP also generates a meal plan while matching to specific conditions of the patient. For example, the MP takes into account input data provided by mobile sensors and crowd-sourced information—e.g., open food facts, nutrition facts, GI values, diabetic recipes, etc. The MP applies suitable algorithms such as a genetic algorithm (GA), a neural network coupled with known algorithms to perform tasks such as ingredient selection, food availability, price compliance, cost-nutrition analysis, dietary compliance verification, and recipe generation.

In an aspect, then, is an apparatus for providing digitized menu options to electronic user devices operated by users, the apparatus comprising: a memory for storing processor-executable instructions and a plurality of accounts each for storing at least historical sensor data of each of the users received by respective ones of the electronic user devices, respectively; a communication interface for receiving an instant sensor data and identification information pertaining to a user sent by a respective one of the electronic user devices, and for transmitting a first digitized menu option to the respective one of the electronic user devices; and a processor, coupled to the memory and the communication interface, for executing the processor-executable instructions in the memory that cause the apparatus to: identify a first of the plurality of accounts stored in the memory based on the identification information, the account associated with the user and for storing historical sensor data pertaining to the user; generate a diabetes risk score for the user based at least on the instant sensor data and historical sensor data; generate a first digitized menu option for the user based on the diabetes risk score; and send a message to the respective one of the electronic user devices, the message configured to cause a display on the respective one of the electronic user devices to modify a user interface and display the first digitized menu option. In embodiments:

the apparatus further comprises a data analysis module configured to generate the diabetes risk score based at least on the instant sensor data, historical sensor data, location, income level, user context, and known risk factors of the user;

the apparatus further comprises a meal planner module configured to determine the first digitized menu option for the user based at least in part on a factor selected from the diabetes risk score; a time; a date; a recipe; an availability of a foodstuff; a market price for a foodstuff, a historic pattern for a recipe; a frequency of recommendation of a recipe; and a frequency of recommendation of a foodstuff;

the communication interface is configured to send and receive information via a distributed network selected from a cellular network, a data network, and a peer-to-peer network;

the instant sensor data are data obtained from a sensor disposed on the respective one of the electronic user devices and wherein the sensor is selected from: a camera sensor, a microphone sensor, a light sensor, a GPS sensor, a motion sensor, gyroscope and accelerometer;

the instant sensor data is data obtained from a sensor disposed on the respective one of the electronic user devices, and wherein the instant sensor data pertains to the speech, gait, facial expression, dietary habit, or level of obesity of the user;

the first digitized menu option comprises a single meal plan, a daily meal plan, or a weekly meal plan, or a user-based configurable meal plan;

the message is configured to initiate an interactive user interface on the respective one of the electronic user devices (or more than one such device);

the message is configured to initiate an interactive user interface on the respective one (or more) of the electronic user devices, and wherein the communication interface is further configured to transmit data to the interactive user interface and receive user input from the interactive user interface via a distributed network; and

the message is configured to initiate an interactive user interface on the respective one (or more) of the electronic user devices, and wherein the communication interface is further configured to transmit data to the interactive user interface and receive user input (selected from text, voice commands, and gestures) from the interactive user interface via a distributed network.

In an aspect is a method for providing digitized menu options to electronic user devices operated by users using the apparatus as above, the method comprising receiving, by the communication interface, instant sensor data from the respective one of the electronic user devices, determining, by the processor, a first digitized menu option, and sending, by the communication interface, the first digitized menu option to the respective one of the electronic user devices.

In an aspect is a method for providing a meal plan to a user by a computer server in communication with electronic user devices, the method comprising: (a) receiving, by the computer server, instant sensor data and identification information pertaining to the user sent by a respective one of the electronic user devices; (b) providing the identification information to a processor within the computer server; (c) identifying, by the processor, an account associated with the user that is stored in a memory coupled to the processor, based on the identification information, the account for storing historical sensor data received from the user in the past; (d) generating a first digitized menu option for display by the respective one of the electronic user devices by the processor by: generating a diabetes risk score for the user based at least on the instant sensor data and historical sensor data; and generate a first digitized menu option for the user based on the diabetes risk score; and (e) sending the first digitized menu option to the respective one of the electronic user devices in a message via a communication interface coupled to the processor, wherein the message is configured to alter a user interface of the respective one of the electronic user devices. In embodiments:

the instant sensor data is obtained via a sensor disposed on the respective one of the electronic user devices, the sensor being configured to measure a health status of the user;

the instant sensor data is obtained via (one or more) sensor(s) disposed on the respective one (or more) of the electronic user devices, the (one or more) sensor(s) may be configured to measure a health status of the user;

the instant sensor data is obtained via a sensor disposed on the respective one of the electronic user devices, the sensor configured to measure a health status of the user using one or more of the following sensors: blood sugar, temperature, weight, risk level, heart rate, etc. in non-invasive manner;

the identification of the user account is based on a combination of one or more methods such as text, voice, gesture, biometric (e.g. fingerprint, face, Iris scan, etc.);

the respective one of the electronic user devices is selected from a mobile phone, tablet, or wearable device;

the first digitized menu option is generated based further on, the time and date, an availability of a foodstuff, a market price for a foodstuff, and a frequency of recommendation of a foodstuff;

the method further comprises notifying a third party based on the diabetes risk score, the third party selected from a second user associated with the user and an emergency service provider;

the message is configured to initiate a voice-or gesture activated interactive user interface on the respective one of the electronic user devices;

the first digitized menu option is generated to reduce the diabetic risk of the user and is selected based on nutrition requirements of the user; and

the method further comprises repeating steps (a)-(e) in order to regulate the diabetes risk score of the user.

In an aspect is a system comprising: a processor; a memory coupled to the processor, the memory configured to store program instructions for instructing the processor to carry out the method as above.

In an aspect is a method for providing advisory services to a user at risk of diabetes, the method comprising: receiving, by a server via a distributed network, an instant sensor data pertaining to a user, the instant sensor data obtained by a sensor disposed on a user device, wherein the instant sensor data provides information as to the diabetic state of the user; accessing, by the server, historical sensor data pertaining to the user, the historical sensor data providing information as to the development of the diabetic state of the user; analyzing, by a data analysis component of the server, the instant sensor data and historical sensor data to determine a diabetes risk score for the user; determining, by a menu planner component of the server, a first digitized menu option based on the determined diabetes risk score for the user; generating, by the server, a message, the message comprising the first digitized menu option; and transmitting, by the server via the distributed network, the message to the user device, the message configured to initiate an interactive user interface on the user device.

In an aspect is an apparatus for providing digitized menu options to an electronic user device operated by a user, the apparatus comprising: a memory for storing processor-executable instructions and an account for storing at least historical sensor data of the user received by the electronic user device; a communication interface for receiving an instant sensor data and identification information pertaining to the user sent by the electronic user device, and for transmitting a first digitized menu option to the electronic user device; and a processor, coupled to the memory and the communication interface, for executing the processor-executable instructions in the memory that cause the apparatus to: identify the account stored in the memory based on the identification information, the account associated with the user and for storing historical sensor data pertaining to the user; generate a diabetes risk score for the user based at least on the instant sensor data and historical sensor data; generate a first digitized menu option for the user based on the diabetes risk score; and send a message to the electronic user device, the message configured to cause a display on the electronic user device to modify a user interface and display the first digitized menu option.

These and other aspects of the invention will be apparent to one of skill in the art from the description provided herein, including the examples and claims.

### BRIEF DESCRIPTION OF THE DRAWINGS

FIG. 1 provides a schematic of interactions within a system according to an embodiment of the invention.

FIG. 2 provides a schematic of a server databases and modules according to an embodiment of the invention.

### DETAILED DESCRIPTION

In an aspect is an apparatus for providing digitized menu options to electronic user devices operated by users, the apparatus comprising: a memory for storing processor-executable instructions and a plurality of accounts each for storing at least historical sensor data of each of the users received by respective ones of the electronic user devices, respectively; an instrumentation and communication interface for receiving one or more instant sensor data and identification information pertaining to a user sent by a respective one of the electronic user devices, and for transmitting a first digitized menu option to the respective one of the electronic user devices; and a processor, coupled to the memory and the communication interface, for executing the processor-executable instructions in the memory that cause the apparatus to: identify a first of the plurality of accounts stored in the memory based on the identification information, the account associated with the user and for storing historical sensor data pertaining to the user; generate a diabetes risk score for the user based at least on the one or more instant sensor data and historical sensor data; generate a first digitized menu option for the user based on the diabetes risk score; and send a message to the respective one of the electronic user devices, the message configured to cause a display on the respective one of the electronic user devices to modify a user interface and display the first digitized menu option.

In an aspect is a method for providing digitized menu options to electronic user devices operated by users using the apparatus as above, the method comprising receiving, by the communication interface, the one or more instant sensor data from the respective one of the electronic user devices, determining, by the processor, a first digitized menu option, and sending, by the communication interface, the first digitized menu option to the respective one of the electronic user devices.

In an aspect is a method for providing a meal plan to a user by a computer server in communication with electronic user devices, the method comprising: (a) receiving, by the computer server, the one or more instant sensor data and identification information pertaining to the user sent by a respective one of the electronic user devices; (b) providing the identification information to a processor within the computer server; (c) identifying, by the processor, an account associated with the user that is stored in a memory coupled to the processor, based on the identification information, the account for storing historical sensor data received from the user in the past; wherein the identification of the user account is based on a combination of one or more of methods such as text, voice, gesture, biometric; (d) generating a first digitized menu option for display by the respective one of the electronic user devices by the processor by: generating a diabetes risk score for the user based at least on the one or more instant sensor data and historical sensor data; and generate a first digitized menu option for the user based on the diabetes risk score; and (e) sending the first digitized menu option to the respective one of the electronic user devices in a message via a communication interface coupled to the processor, wherein the message is configured to alter a user interface of the respective one of the electronic user devices. In embodiments, the method further comprises repeating steps (a)-(e) in order to regulate the diabetes risk score of the user.

In an aspect is a method for providing advisory services to a user at risk of diabetes, the method comprising: receiving, by a server via a distributed network, a one or more instant sensor data pertaining to a user, the one or more instant sensor data obtained by a one or more sensors deployed or embedded on a one or more user devices, wherein the one or more instant sensor data provide information pertaining to the diabetic state of the user; accessing, by the server, historical sensor data pertaining to the user, the historical sensor data providing information as to the development of the diabetic state of the user; analysing, by a one or more data analysis components of the server, the one or more instant sensor data and historical sensor data to determine a diabetes risk score for the user; determining, by a menu planner component of the server, a first digitized menu option based on the determined diabetes risk score for the user; generating, by the server, a message, the message comprising the first digitized menu option; and transmitting, by the server via the distributed network, the message to the user device, the message configured to initiate an interactive user interface on the user device.

In aspects are system(s) configured to carry out the methods described herein. The system comprises a processor and a memory coupled to the processor, the memory configured to store program instructions for instructing the processor to carry out the method. Further details are provided below. It will be appreciated, however, that certain components of such systems, and further certain steps of the associated methods, may be omitted from this disclosure for the sake of brevity. The omitted components and steps, however, are merely those that are routinely used in the art and would be easily determined and implemented by those of ordinary skill in the art using nothing more than routine experimentation. Throughout this specification, where hardware is described, it will be assumed that the devices and methods employing such hardware are suitably equipped with necessary software (including any firmware) to ensure that the devices/methods are fit for the described purpose.

In embodiments, the systems and methods involve electronic user devices operated by or configured for operation by a user. The user device may be, for example, a mobile user device, a wearable user device, a fixed position user device, or a combination thereof. Examples of mobile user devices include mobile phones, tablets, laptops, personal digital assistants, and the like. Examples of wearable devices include smart watches, personal fitness monitors, and the like. Examples of fixed position devices include desktop computers and devices installed or configured to be installed in a vehicle. The user device comprises a variety of components including a user interface (e.g., a visual interface such as a touch-screen and camera, an audio interface such as a microphone/speaker pair, etc.), a communications module, a sensor, a power source, etc. The device may further comprise a variety of optional components such as a memory for storing sensor data, a processor, etc.

In embodiments, the user device comprises a sensor, and may include a plurality of sensors such as 2, 3, 4, 5, or more than 5 sensors. The sensors may be embedded in the device or peripheral to the device, or a combination thereof. Examples of sensors include a camera sensor, a microphone sensor, a light sensor, a GPS sensor, a motion sensor, a gyroscope, and an accelerometer. The sensor is configured to obtain instant sensor data, and such instant sensor data may be stored locally (for later transmission as data packets) and/or transmitted in real time. Transmission may be, e.g., via the communications module of the user device using a distributed network. Instant sensor data may be tagged by the sensor with appropriate metadata, such as the time and location of acquisition of the data, or such metadata may be appended to the instant sensor data by another component of the user device or by another component of the systems herein. The sensor is disposed on/in (i.e., deployed, embedded, attached, or otherwise associated with) the user device.

In embodiments, the user device comprises a communications module. The communications module may be any suitable, including a Bluetooth, Wifi, GSM, or other component for communicating with a distributed network, particularly via wireless communication with the distributed network. Examples of distributed networks include a cellular network, a data network, and a peer-to-peer network, among others. The communication interface is configured to send and receive information via the distributed network. Such information includes instant sensor data, instructions and messages from a server, data suitable as metadata, and the like. The communications module includes an interface for interacting with other components of the user device (e.g., the user interface).

In embodiments, the instant sensor data are data that pertain to the speech, gait, facial expression, dietary habit, health status, or level of obesity of the user, or any other aspect of the user or the user's activities that may be usable by the system in the methods herein. For example, in embodiments the sensors are configured to measure a health status of the user using one or more of the following blood sugar, temperature, weight, risk level, heart rate, etc. Such data may be obtained from a single sensor or a plurality of sensors working in cooperation. In embodiments the sensor is configured for non-invasive measurement and collection of instant sensor data. The instant sensor data may be augmented with appropriate and desired metadata, such metadata selected from any combination of the time and date of measurement, the location of the measurement, the identity of the user device, the identity of the user, and the like.

Using the communications module, the user device transmits the instant sensor data (either in real time or as batches of data, or a combination thereof) via a distributed network to be received by a server. The server receives the instant sensor data and associates it with a user account. The association can be made in any appropriate way. For example, the instant sensor data can be labelled with metadata that includes an identification number/label assigned to the user. The user account may also be identified based on a combination of one or more of methods such as text, voice, gesture, biometric (e.g., fingerprint, face, Iris, etc.). Alternatively, the server can detect and determine the originating user device for the incoming instant sensor data, and then determine the user associated with the specific user device. Ultimately, the server determines the user associated with the instant sensor data and accesses the user account. Each user creates or is given a user account on the server, and such account is used to store, track, and maintain historical data, activity, and relevant information pertaining to the user. Such information is used as described herein in various determinations performed by the server. The instant sensor data may continue to be referred to herein as instant sensor data even if that data is not current or recently obtained. For example, the server keeps the instant sensor data in the user account and such data becomes historical sensor data as new instant sensor data is obtained. Nevertheless, all such data may alternatively be referred to herein as instant sensor data.

In embodiments the server comprises a variety of modules and components. For example, the server comprises an instrumentation and communication interface (also referred to herein as a communication module) configured to communicate with user devices and other devices using a distributed network. In embodiments the interface is configured for receiving one or more instant sensor data and identification information pertaining to a user sent by a user device. In embodiments the interface is configured for transmitting messages such as a message comprising a digitized menu option and other information to the user device.

In embodiments the server further comprises one or more data analysis modules configured to generate the diabetes risk score. Determination of the diabetes risk score can be based on any appropriate measured data, information, and algorithm provided that the determination results in a diabetes risk score consistent with the intentions/goals/activities described herein. For example, in embodiments the server determines a diabetes risk score based at least on one or more of the following: instant sensor data, historical sensor data, location (i.e., instant location of the user, residence location of the user, etc.), income level (i.e., income of the user), user context (e.g., personal characteristics of the user, likes and dislikes identified by/for the user, etc.), and known risk factors of the user (i.e., risk factors for diabetes such as family history and the like). A variety of algorithms known in the prior art (e.g. US 20130332082 A1, US 20120309030 A1) can be used by the data analysis module (or a plurality of data analysis modules) for determining the diabetes risk score from the data mentioned above and other data/information as appropriate.

In embodiments the server comprises a meal planner module configured to determine a first digitized menu option for the user based at least in part on a factor selected from: the diabetes risk score (i.e., as determined by the data analysis module); a time (i.e., a current time, or the time associated with selected instant sensor data); a date (i.e., a current date, or the date associated with selected instant sensor data); a recipe; an availability of a foodstuff; a market price for a foodstuff, a historic pattern for a recipe; a frequency of recommendation of a recipe; a frequency of recommendation of a foodstuff; an availability of one or more recipes for a meal; a market price for the one or more recipes for a meal; and a historic pattern or frequency of recommendation of a the one or more recipes for a meal. In addition to the first digitized menu option the meal planner module may further determine a plurality of additional digitized menu options, each of which can be communicated to the user in the same manner and at the same time, or over a predetermined period of time, such as according to a communication schedule. The schedule can be selected (and continuously updated, if necessary) to ensure, for example, that the user has a recommended menu option at selected times throughout the hours of the day or days of the week.

In embodiments, the first digitized menu option comprises a single meal plan, a daily meal plan, or a weekly meal plan. Each such plan may comprise one or more recipes, one or more ingredients lists, one or more reference materials and/or cooking aids (e.g., instructional videos, professional cooking tips, etc.), and the like. Each such plan may further comprise information specific to the user and/or specific to the diabetes risk level for the user. In embodiments the first digitized menu option is generated to reduce the diabetic risk of the user and is selected based on nutrition requirements of the user, among other possible factors.

The first digitized menu option (as well as further menu options and other information as described herein) is communicated to the user device and to the user. Accordingly, the server is configured to communicate a message to the user device. In embodiments, the message is configured to cause a user interface of the user device to be altered, specifically to communicate the menu option and any other information to the user. In embodiments, the message is configured to cause a display on the user device to modify a user interface and display the first digitized menu option. In embodiments, the message is configured to initiate an interactive user interface on the user device. In embodiments, the message is configured to initiate an interactive user interface on the user device. In embodiments the communication module/interface is further configured to transmit data to the interactive user interface and receive user input from an interactive user interface via a distributed network. In embodiments, the user interface is based on text, image, voice, and/or gesture enabled interfaces. In embodiments, the message is configured to initiate a voice- or gesture-activated interactive user interface on the user device. The message is communicated to the user device via any suitable method of communicating given the context—e.g., the type of distributed network(s) available to the server and user device at the moment, the content of the menu option and information to be communicated, etc.

The message described above and herein is an output of the server. A further output may include a notification (also referred to herein as an amelioration action). An example of a notification includes where the server notifies a third party based on the diabetes risk score, the third party selected from a second user associated with the user and an emergency service provider. Examples of third parties include ambulances and other emergency services, known relatives of the user, friends of the user, medical professionals such as a primary care physician of the user, and the like. These contacts can be specified ahead of time by the user and stored in the user profile.

Furthermore in an aspect is a method and system comprising: an instrumentation and monitoring user device (e.g. sensor, wearable, mobile, tablet, watch, etc.); an intelligent meal planner (MP); a module and/or component for detecting or predicting the risk level for a diabetic patient from data pertaining to a factor selected from speech, gait, facial expression, dietary habit, level of obesity, and the like; and based on said detection or prediction and blood glucose levels, a component for the MP to generate optimal meal plan to regulate said diabetic state. In embodiments, the user device includes one or more sensors (e.g. blood, temperature, weight scale, height, heart rate), and may be a mobile phone, tablet, wearable for measuring, monitoring or transmitting the person's heath status (e.g. measuring blood sugar, weight, risk level). The risk analysis is based on a reasoning algorithm on the sensory data, past history (individual, cohorts or those connected in a social network) and/or context of the person, along with other optional factors. The risk analysis, for example, returns value of any of: high, medium, low, and minor, wherein for each risk level, the required minimum nutrition is recommended. Alternatively, the risk level can be a numerical value.

In embodiments the method disclosed herein includes determining or predicting diabetic signals: using deep learning techniques to detect change in diabetic state from speech pattern, facial expression, heart rate etc.; determining dietary or nutrition requirements of the person based on said risk level, his or her glucose level and health profile; and estimating or determining the optimal time needed to adjust the said health risk profile. In embodiments, the method for the detecting or predicting may communicate with the patient through voice command or alerting the patient's family member or friends or ambulance depending on the severity level.

In embodiments the method for the meal planner (MP) is based on: recipes or ingredients that matches seasonal, regional and budget constraints of the person based on cohort (e.g., market information, weather data) and analysis (e.g., patient's personal and family history such as allergy, other medical conditions), as well as cultural and religious dietary rules and restrictions; use of various optimization functions, such as an algorithm, that takes into consideration various factors such as dietary or nutrition requirement, medication used, required time T to regulate the risk, and other conditions.

In embodiments the method for generating the meal plan may also predict the next menu (including when to eat or drink and in what portions, and at what time) for the person.

In embodiments the method as applied in a mobile system may be a platform for the person to compose a meal menu based on recommended ingredients and recipes, as well as a platform for the person to keep track his or her eating history.

In embodiments the method can further integrate education materials for the person, wherein the user interaction, sentiment and engagement may be analysed using video-based analytics (e.g., of stress level of the user from facial expression). In embodiments the method can allow users to exchange information based on one user's experience, or to share with physician to enable community support and synchronization with in-hospital visits. In embodiments the method can generate a health report based on analytics models built based ingested health guidelines or scorecard for the user based on dietary progress to date, etc.

In embodiments the method can use existing software to crawl existing market or farm information systems and generates alerts to the person. The meal plan generation algorithm may update the user preference based on this result.

Various embodiments of the invention are described more fully hereinafter with reference to the accompanying drawings. The invention herein may be embodied in many different forms and should not be construed as limited to the embodiments set forth in the drawings; rather, these embodiments are provided to provide further illustrative non-limiting examples. Arrowheads in the figures are provided merely as examples of directions for the flow of data but are not exhaustive and are not meant to be limiting—i.e., data may flow (where appropriate) in directions that are not shown by arrowheads in the figures. Similar numbers in different figures are meant to refer to similar components.

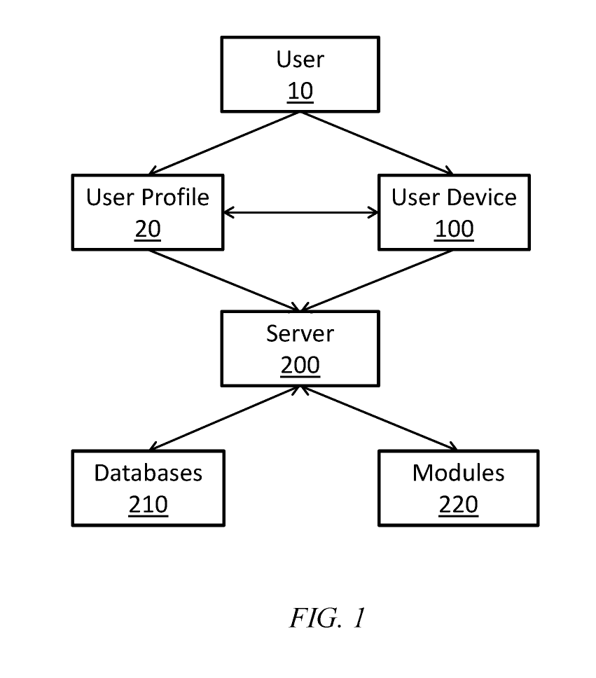

With reference to FIG. 1, there is shown a schematic of interactions within a system according to an embodiment of the invention. User 10 uses User Device 100 and provides some or all of the information contained in User Profile 20 via User Device 100. Other information in User Profile 20 may be obtained automatically and from a variety of sources (e.g., via accessing various online databases—public and/or private (the latter with appropriate consent and/or other appropriate privacy protections)—such as social media accounts, health records, and the like). User Device 100 interacts with Server 200, which may also store User Profile 20. Server 200 comprises one or more Databases 210 and the Modules 220 as described herein. The User Profile 20 may include goals, preferences, medication, vital statistics, and the like for User 10. The User Device 100 may include one or more sensors, and may comprise a processor and memory for carrying out processing and/or storage of data. A copy of the User Profile 20 may be kept on the User Device 100.

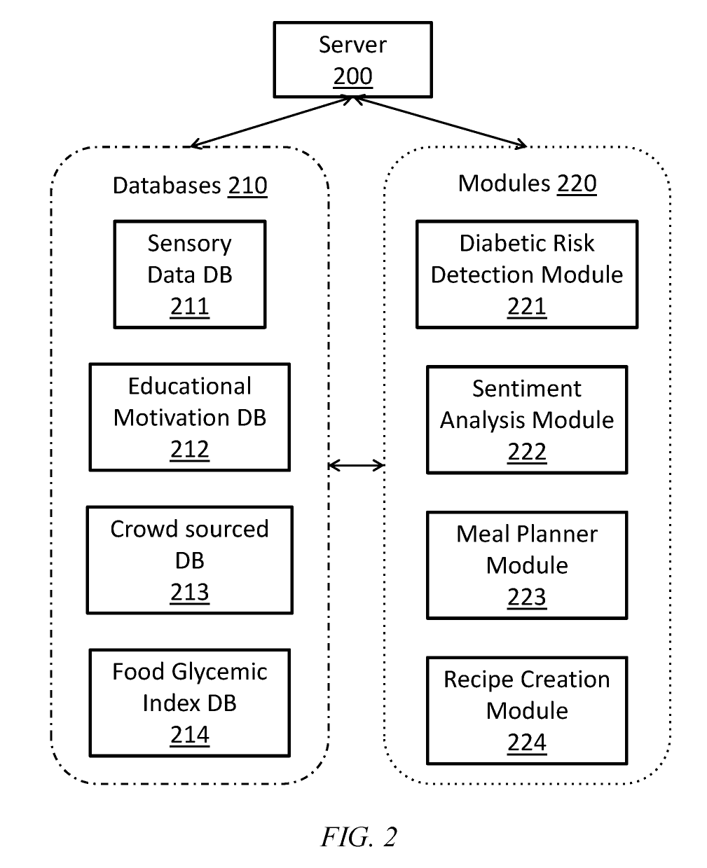

With reference to FIG. 2, there is provided a schematic of a server and the databases and modules contained therein according to an embodiment of the invention. Within Server 200, the Databases 210 component includes but is not limited to the following. Sensory data database 211 includes data obtained from sensors on user devices. This may include a variety of data types and formats including text, video, statistics, and the like. Educational motivation database 212 includes general educational materials, general motivational information and references, and the like. Crowd sourced database 213 includes data and information obtained non-specifically from users and other interested parties. This may include food prices, recipes, and the like. Food glycemic index database 214 includes general information about the glycemic index of known foods and combinations of foods (e.g., menus, recipes, etc.).

Within Server 200, the Modules 220 component includes but is not limited to the following. The Diabetic Risk Detection Module 221 uses sensor data such as data collected on speech, gait, facial expressions, etc. as described herein to determine a diabetes risk score. The Sentiment Analysis Module 222 may be used to determine the sentiment of a user based on various sensor data, historical data, and/or context data. The Meal Planner Module 223 determines suitable meals, recipes, and/or recommendations based on the diabetic risk score, sentiment analysis, and/or contextual data. The Recipe Creation Module 224, uses, for example a cognitive algorithm for determining new recipes suitable for a variety of situations/users/contexts. An example of this module is based on the IBM® Watson Chef technology.

It will be appreciated that the server may be a single stand-alone server or may be a cloud-based (or otherwise de-localized) system that accesses various resources from various locations.

Throughout this disclosure, use of the term “server” is meant to include any computer system containing a processor and memory, and capable of containing or accessing computer instructions suitable for instructing the processor to carry out any desired steps. The server may be a traditional server, a desktop computer, a laptop, or in some cases and where appropriate, a tablet or mobile phone. The server may also be a virtual server, wherein the processor and memory are cloud-based.

The methods and devices described herein include a memory coupled to the processor. Herein, the memory is a computer-readable non-transitory storage medium or media, which may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Throughout this disclosure, use of the term “or” is inclusive and not exclusive, unless otherwise indicated expressly or by context. Therefore, herein, “A or B” means “A, B, or both,” unless expressly indicated otherwise or indicated otherwise by context. Moreover, “and” is both joint and several, unless otherwise indicated expressly or by context. Therefore, herein, “A and B” means “A and B, jointly or severally,” unless expressly indicated otherwise or indicated otherwise by context. Furthermore, use of singular terms such as “a”, “an”, and “the” are meant to include, unless indicated expressly or by context, situations of the plural. Thus, for example, reference to “a processor” includes situations where two or more processors (i.e., a plurality of processors) are used, and reference to a device with “a user interface” includes devices that have a plurality of user interfaces.

It is to be understood that while the invention has been described in conjunction with examples of specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. The pertinent parts of all publications mentioned herein are incorporated by reference. All combinations of the embodiments described herein are intended to be part of the invention, as if such combinations had been laboriously set forth in this disclosure.

### EXAMPLES

User X is a 40-year old woman living in a developing region with her family including three children. She was born 26.02.1976. She is exhibiting the following symptoms: fatigue and weight gain causing her to be ineffective at home and work. She remembers previous blood glucose test prior to birth of children was high, however she didn't act on it. She is currently under work and family stress.

After a visit to the doctor, User X is diagnosed with Type 2 diabetes using the A1C test. She is told to lose weight over the next 3 months. She is given the medicines: Glimepiride, Victoza and Metformin. Frustrated and not wanting to ignore the current situation, she seeks the assistance of the system and methods described herein.

She obtains a device (also referred to as a regulator) with a sensor and communication module able to communicate with a remote server configured as described herein. The non-invasive sensors in the regulator allow User X to get a daily blood glucose reading. They also compute her diabetic state and risk i.e. low, medium, high, using her facial expression, speech pattern, heart rate etc. (or the device communicates the data to a server capable of such determination).

She inputs her medication list and the dosage, as well as current weight and target weight. The regulator asks for inputs of her family size and their demographic, as well as her monthly budget. Alternatively, the regulator may obtain her current weight from readily available BMI kiosks, and similarly the target weight can be predicted based on historic weight data and planned meals and activities.

Her risk level is shared with her on her mobile/wearable device, including support such as ‘User X you try today, your glucose don reduce’—i.e., a statement that is tailored to the lingua franca in Abuja (her developing region home).

The regulator offers daily meal plans based on the prices of foods in Abuja and given that it is the dry season. It uses the GI of foods to recommend meals that minimize spikes in blood glucose levels while ensuring her health is maintained. The regulator also optimizes for nutritious food suitable for her family.

The ingredients are shared with User X, and the regulator proposes some Nigerian inspired recipes that she can prepare for her family.

In addition to meal planning, the regulator uses her facial expression, heart rate, etc. to gauge her sentiment and provide appropriate daily encouragement e.g. ‘If you walk to location X in Abuja, your glucose will drop to a safe level.’

The regulator stores her daily glucose and risk level and upon User X's 3-month medical check-up, she shares the data with her physician. Through the regulator, User X's blood sugar level has started decreasing and her doctor agrees she is on the right path. Her monthly budget has not been exceeded and the health of her family is not compromised. Some specific data and recommendations are provided below.

User X's State at time t prior to next meal

Hyperglycemic event: 168 mg/dL—obtained from non-invasive glucose monitor

Risk Level: Warning—risk level prediction

Medication: Victoza, Metformin, Glimepiride

Meal Preferences

Carbohydrate constraints: Does not like potatoes

Protein constraints: No pork due to religious reasons

Fat constraints: None

Vegetable constraints: Prefers carrots and spinach

Goals: Weight loss of 6 lbs over next 3 months.

MP Optimization with Genetic Algorithm

Hyperglycemic event: MP goal is to provide nutritious meal without further raising glucose level.

High glucose level and high risk indicate need for foods with low GI.

MP identifies carbohydrates that are in season in Abuja and are within budget. It eliminates potatoes.

MP initializes with random subset [Carbohydrates, protein, vegetable, oil] and calculates a fitness score. The fitness score accounts for current risk and glucose level, target glucose level, nutritional constraints, price, preferences.

The fitness score is improved on each iteration through mutations [substitution with different foods] until the desired fitness value is crossed.

Ingredients are then shared with User X and Nigerian inspired recipes are recommend.

The system further uses a cognitive online algorithm—IBM® Chef Watson—to develop and recommend recipes in the meal plan.

What is claimed is:

1. A method for providing digitized menu options to electronic user devices operated by users, using an apparatus for providing digitized menu options, the method comprising
operating said apparatus for providing digitized menu options, the apparatus comprising:
a memory for storing processor-executable instructions and a plurality of accounts each for storing at least historical sensor data of each of the users received by respective ones of the electronic user devices, respectively;
a communication interface for receiving an instant sensor data and identification information pertaining to a user sent by a respective one of the electronic user devices, and for transmitting a first digitized menu option to the respective one of the electronic user devices; and
a processor, coupled to the memory and the communication interface, for executing the processor-executable instructions in the memory that cause the apparatus to:
identify a first of the plurality of accounts stored in the memory based on the identification information, the account associated with the user and for storing historical sensor data pertaining to the user;
generate a diabetes risk score for the user based at least on the instant sensor data and historical sensor data;
generate a first digitized menu option for the user based on the diabetes risk score; and
send a message to the respective one of the electronic user devices, the message configured to cause a display on the respective one of the electronic user devices to modify a user interface and display the first digitized menu option;
receiving, by the communication interface, the instant sensor data from the respective one of the electronic user devices,
determining, by the processor, the first digitized menu option, and
sending, by the communication interface, the first digitized menu option to the respective one of the electronic user devices.

2. A method for providing a meal plan to a user by a computer server in communication with electronic user devices, the method comprising:
(a) receiving, by the computer server, instant sensor data and identification information pertaining to the user sent by a respective one of the electronic user devices;
(b) providing the identification information to a processor within the computer server;
(c) identifying, by the processor, an account associated with the user that is stored in a memory coupled to the processor, based on the identification information, the account for storing historical sensor data received from the user in the past;
(d) generating a first digitized menu option for display by the respective one of the electronic user devices by the processor by: generating a diabetes risk score for the user based at least on the instant sensor data and historical sensor data; and generate a first digitized menu option for the user based on the diabetes risk score; and
(e) sending the first digitized menu option to the respective one of the electronic user devices in a message via a communication interface coupled to the processor, wherein the message is configured to alter a user interface of the respective one of the electronic user devices.

3. The method of claim 2, wherein the instant sensor data is obtained via a sensor disposed on the respective one of the electronic user devices, the sensor configured to measure a health status of the user.

4. The method of claim 2, wherein the respective one of the electronic user devices is selected from a mobile phone, tablet, or wearable device.

5. The method of claim 2, wherein the first digitized menu option is generated based further on, the time and date, an availability of a foodstuff, a market price for a foodstuff, and a frequency of recommendation of a foodstuff.

6. The method of claim 2, wherein the method further comprises notifying a third party based on the diabetes risk score, the third party selected from a second user associated with the user and an emergency service provider.

7. The method of claim 2, wherein the message is configured to initiate a voice-or gesture activated interactive user interface on the respective one of the electronic user devices.

8. The method of claim 2, wherein the first digitized menu option is generated to reduce the diabetic risk of the user and is selected based on nutrition requirements of the user.

9. The method of claim 2, wherein the method further comprises repeating steps (a)-(e) in order to regulate the diabetes risk score of the user.

10. A method for providing advisory services to a user at risk of diabetes, the method comprising:
receiving, by a server via a distributed network, an instant sensor data pertaining to a user, the instant sensor data obtained by a sensor disposed on a user device, wherein the instant sensor data provides information as to the diabetic state of the user;
accessing, by the server, historical sensor data pertaining to the user, the historical sensor data providing information as to the development of the diabetic state of the user;
analyzing, by a data analysis component of the server, the instant sensor data and historical sensor data to determine a diabetes risk score for the user;
determining, by a menu planner component of the server, a first digitized menu option based on the determined diabetes risk score for the user;
generating, by the server, a message, the message comprising the first digitized menu option; and
transmitting, by the server via the distributed network, the message to the user device, the message configured to initiate an interactive user interface on the user device.
